(19) United States
(12) Patent Application Publication
(10) Pub. No.: US 2008/0311604 A1
(43) Pub. Date: Dec. 18, 2008

(54) Methods for Prediction and Prognosis of Cancer, and Monitoring Cancer Therapy

(76) Inventors: James J. Elting, Madison, CT (US); Walter P. Carney, North Andover, MA (US); Peter J. Hamer, Reading, MA (US); Douglas Bigwood, Madison, CT (US)

Correspondence Address: LEONA L. LAUDER, 235 MONTGOMERY STREET, SUITE 1026, SAN FRANCISCO, CA, 94104-0332, US

(21) Appl. No.: 12/091,899

(22) PCT Filed: Nov. 1, 2006
(86) PCT No.: PCT/US06/42661

§ 371 Date: Aug. 1, 2008

Related U.S. Application Data
(60) Provisional application No. 60/733,098, filed on Nov. 2, 2005.

(51) Int. Cl. G01N 33/53; C12Q 1/00

(52) U.S. Cl. 435/7.94; 435/4

(57) ABSTRACT

The present invention relates to biomarkers and the use of biomarkers for the prediction and prognosis of cancer as well as the use of biomarkers to monitor the efficacy of cancer treatment. Specifically, this invention relates to the use of soluble VEGF-R2 as a biomarker for multi-kinase inhibitors.

### FIELD OF THE INVENTION

The present invention relates to biomarkers and the use of biomarkers for the prediction and prognosis of cancer as well as the use of biomarkers to monitor the efficacy of cancer treatment. Specifically, this invention relates to the use of soluble VEGF-R2 as a biomarker for multi-kinase inhibitors.

### BACKGROUND OF THE INVENTION

Vascular endothelial growth factor receptors (VEGFRs) and their ligands, vascular endothelial growth factors (VEGFs), play critical roles in endothelial cell migration and proliferation. The VEGFR/VEGF system includes three receptors (VEGFR-1, VEGFR-2, and VEGFR-3) and four ligands (VEGF-A, B, C, D, and E and placental growth factor). VEGF-A further consists of four isoforms, VEGF-121, VEGF-165, VEGF-185, and VEGF-204, derived from alternative transcription of the VEGF-A gene. The receptors are plasma membrane-spanning proteins with intracellular tyrosine kinase domains. As with other protein kinases, activation of the VEGFRs is a key mechanism in regulating signals for endothelial cell proliferation, and abnormalities of VEGFR/VEGF are thought to contribute to abnormal angiogenesis in number of human diseases such as psoriosis and malignancy.

In embryogenesis, the VEGFR/VEGF system is essential for the correct development of the vascular system. In adults, VEGFR/VEGF is important in wound healing, inflammation, and angiogenesis.

A noninvasive assay for circulating soluble VEGF-R2 levels in patients prior to drug treatment is a potentially important adjunct to therapeutic decision making. Although assays of total VEGF-A have been used in humans as a prognostic indicator of disease outcome, until the instant disclosure, no correlation between levels of soluble VEGF-R2 in patients prior to chemotherapy and treatment outcome have been reported. Therefore, soluble VEGF-R2 may serve as a valuable prognostic indicator, and as a biomarker to monitor the efficacy of treatment with a multi-kinase inhibitor.

### SUMMARY OF THE INVENTION

The present invention relates to biomarkers and the use of biomarkers for the prediction and prognosis of cancer as well as the use of biomarkers to monitor the efficacy of cancer treatment. Specifically, this invention relates to the use of soluble VEGF-R2 as a biomarker for a multi-kinase inhibitor (e.g., Sorafenib).

In one embodiment, the present invention relates to the use of quantitative immunoassays to measure levels of soluble VEGF-R2 protein in human body fluids prior to treatment with a multi-kinase inhibitor (e.g., Sorafenib). Said levels are particularly useful as an indicator of the potential for cancer patients treated with a multi-kinase inhibitor (e.g., Sorafenib) to benefit from such therapy.

Measurement of post-treatment levels of soluble VEGF-R2, as well as the change in soluble VEGF-R2 levels over the course of treatment, can be used clinically as a therapeutic aid for patient therapy selection, to monitor the status of a preneoplastic/neoplastic disease in a patient, and/or to monitor how a patient with a preneoplastic/neoplastic disease is responding to a therapy. In one embodiment, the levels of soluble VEGF-R2 may be used to aid in patient therapy selection, and to make decisions about the optimal method for patient therapy.

The levels of soluble VEGF-R2 may be measured in patient samples such as, but not limited to, blood, serum, plasma, urine, saliva, semen, breast exudate, cerebrospinal fluid, tears, sputum, mucous, lymph, cytosols, ascites, pleural effusions, amniotic fluid, bladder washes, and bronchioalveolar lavages.

In another embodiment, the invention relates to the use of an immunoassay as a method of selecting patients who are likely to benefit from multi-kinase inhibitor (e.g., Sorafenib) treatment by measuring pretreatment levels of soluble VEGF-R2 in patient samples and assessing probable outcome based on a nomogram of likely patient outcome versus soluble VEGF-R2 levels.

A method of monitoring the status of a disease associated with an activated VEGF pathway in a patient may be further prognostic for a disease, wherein the levels of total VEGF protein in the patient's samples are indicative of a better or poorer treatment outcome for the patient. The prognosis may be a clinical outcome selected from the group consisting of response rate (RR), complete response (CR), partial response (PR), stable disease (SD), clinical benefit [including complete response (CR), partial response (PR), and stable disease (SD)], time to progression (TTP), progression free survival (PFS), and overall survival (OS).

These methods may be in standard formats, for example, an immunoassay in the form of a sandwich immunoassay, such as a sandwich enzyme-linked immunosorbent assay (ELISA) or an equivalent assay. These immunoassays may use monoclonal antibodies, such as anti-soluble VEGF-R2 monoclonal antibodies. Furthermore, the monoclonal antibody may be biotinylated.

Another embodiment of the invention relates to a quantitative immunoassay to measure serial changes in the levels of total soluble VEGF-R2 protein in patient samples, as a method of therapy selection for a patient with a disease, for example, a preneoplastic/neoplastic disease.

As an example, one such method of therapy selection may comprises the steps of:
(a) immunologically detecting and quantifying the level of total soluble VEGF-R2 protein in a sample from a control population;
(b) immunologically detecting and quantifying the level of total soluble VEGF-R2 protein in samples taken from a patient over time; and
(c) determining whether to use conventional therapy and/or multi-kinase inhibitor (e.g., Sorafenib) therapy to treat the patient based the level of soluble VEGF-R2 protein in the patient's samples.

For example, if the level of soluble VEGF-R2 protein in a patient's sample is found to be above 70 pg/ml, the conclusion could be drawn that the patient has a soluble VEGF-R2 driven disease, and the decision may be made to use multi-kinase inhibitor (e.g., Sorafenib) therapy to treat the patient, either alone or in conjunction with one or more other therapies.

A soluble VEGF-R2 pathway-directed therapy may be multi-kinase inhibitors, tyrosine kinase inhibitors, bis-aryl ureas, antisense inhibitors of VEGFR-2, or monoclonal antibody therapies, or the like. For example, a VEGF pathway-directed therapy may be the bis-aryl urea Sorafenib, which is an angiogenesis inhibitor as well as a tyrosine kinase inhibitor, or the tyrosine kinase inhibitor, STI571 (also known as imatinib mesylate or Gleevec®).

Another embodiment of the invention relates to the use of quantitative immunoassays to detect changes in soluble VEGF-R2 levels in combination with the levels of one or more other protein(s). Such additional protein(s) may include, for example, inhibitors (e.g., tissue-inhibitor of metalloproteinase-1 (TIMP-1)), oncoproteins (e.g., HER-2/neu, ras p21), growth factor receptors (e.g., epidermal growth factor receptor (EGFR), platelet derived growth factor receptor alpha (PDGFR-α)), metastasis proteins (e.g., urokinase-type plasminogen activator (uPA)), tumor markers (e.g., carcinoembryonic antigen (CEA)), and tumor suppressors (e.g., p53). These methods may then be used, for example, as diagnostic/prognostic tools, therapy selection for patients with a disease, monitoring the status of a disease in a patient, and monitoring how a patient with a disease is responding to a VEGF pathway-directed or other therapy. It would be advantageous to test patients (e.g., cancer patients) for serial changes in both total soluble VEGF-R2 and additional proteins, such as proteins that activate the VEGF pathway, as a means to enlarge the clinical perspective, therapeutic resources, and diagnostic/prognostic parameters in order to select the optimal therapeutic combinations for the most promising treatment outcomes.

In another embodiment, the invention provides a test kit for monitoring the efficacy of a therapeutic in a patient sample, comprising an antibody specific for a protein. In certain embodiments, the kit further includes instructions for using the kit. In certain embodiments, the kit may further include solutions for suspending or fixing the cells, detectable tags or labels, solutions for rendering a polypeptide susceptible to the binding of an antibody, solutions for lysing cells, or solutions for the purification of polypeptides. In a still further embodiment, the antibody is specific for soluble VEGF-R2.

### DESCRIPTION OF THE FIGURES

FIG. 1 illustrates the mean soluble VEGF-R2 levels in patient populations at baseline (pretreatment) and during treatment.

### DETAILED DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

For convenience, the meaning of certain terms and phrases employed in the specification, examples, and appended claims are provided below.

The term “patient sample,” as used herein, refers to a sample obtained from a patient. The sample may be of any biological tissue or fluid. The sample may be a sample which is derived from a patient. Such samples include, but are not limited to, blood, serum, plasma, urine, saliva, semen, breast exudate, cerebrospinal fluid, tears, sputum, mucous, lymph, cytosols, ascites, pleural effusions, peritoneal fluid,amniotic fluid, bladder washes, and bronchioalveolar lavages, blood cells (e.g., white cells), tissue or biopsy samples (e.g., tumor biopsy), or cells therefrom. Biological samples may also include sections of tissues such as frozen sections taken for histological purposes.

The term “biomarker” encompasses a broad range of intra- and extra-cellular events as well as whole-organism physiological changes. Biomarkers may be represent essentially any aspect of cell function, for example, but not limited to, levels or rate of production of signaling molecules, transcription factors, metabolites, gene transcripts as well as post-translational modifications of proteins. Biomarkers may include whole genome analysis of transcript levels or whole proteome analysis of protein levels and/or modifications.

A biomarker may also refer to a gene or gene product which is up- or down-regulated in a compound-treated, diseased cell of a subject having the disease compared to an untreated diseased cell. That is, the gene or gene product is sufficiently specific to the treated cell that it may be used, optionally with other genes or gene products, to identify, predict, or detect efficacy of a small molecule. Thus, a biomarker is a gene or gene product that is characteristic of efficacy of a compound in a diseased cell or the response of that diseased cell to treatment by the compound.

The term “cancer” includes, but is not limited to, solid tumors, such as cancers of the breast, respiratory tract, brain, reproductive organs, digestive tract, urinary tract, eye, liver, skin, head and neck, thyroid, parathyroid, and their distant metastases. The term also includes lymphomas, sarcomas, and leukemias.

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumor.

Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus.

Tumors of the digestive tract include, but are not limited to, anal, colon, colorectal, esophageal, gallbladder, gastric, pancreatic, rectal, small-intestine, and salivary gland cancers.

Tumors of the urinary tract include, but are not limited to, bladder, penile, kidney, renal pelvis, ureter, and urethral cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal/hypopharyngeal/nasopharyngeal/oropharyngeal cancer, and lip and oral cavity cancer.

Lymphomas include, but are not limited to, AIDS-related lymphoma, non-Hodgkin's lymphoma, cutaneous T-cell lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “patient” or “subject” as used herein includes mammals (e.g., humans and animals).

The present invention is directed to quantitative immunoassays that measure the levels of soluble VEGF-R2 protein in patient samples. These assays may be useful for the selection of a therapy for a patient with a disease associated with an activated soluble VEGF-R2 pathway. As used herein, an “activated VEGF pathway” is defined as a VEGF pathway activated by either overexpression or mutation of soluble VEGF-R2 protein and as such, encompasses upregulated and/or mutationally stimulated VEGF pathways.

Examples of neoplastic diseases associated with an activated VEGF pathway, as well as precancers leading to neoplastic diseases, are the following: metastatic medulloblastoma, gastrointestinal stromal tumors (GIST), dermatofibrosarcoma protruberans (DFSP), chronic myeloproliferative diseases (CMPD), colorectal cancer, colon cancer, lung cancer, non-small-cell lung cancer, small-cell lung cancer, acute myelogenous leukemia, thyroid cancer, pancreatic cancer, bladder cancer, kidney cancer, melanoma, breast cancer, prostate cancer, ovarian cancer, cervical cancer, head-and-neck cancer, brain tumors, hepatocellular carcinoma, and hematologic malignancies. Thus, the levels of soluble VEGF-R2 protein, alone or in combination with levels of other proteins (e.g., other oncoproteins) may be used to predict clinical outcome and/or as an aid in therapy selection.

Thus, the present invention discloses and claims the application of an immunoassay to quantitatively measure soluble VEGF-R2 levels in patient samples (e.g., circulating soluble VEGF-R2 levels) in order to assess the likelihood that a patient suffering from cancer would benefit from treatment with a multi-kinase inhibitor (e.g., Sorafenib).

In one embodiment of the invention, soluble VEGF-R2 protein is quantitated in patient samples drawn at the time of diagnosis (e.g., renal cell carcinoma), as well as subsequent time points post-treatment (e.g., day 31 of the first cycle of treatment, day 1 of the third cycle of treatment). Such patient samples may be, for example, blood, serum, plasma, urine, saliva, semen, breast exudate, cerebrospinal fluid, tears, sputum, mucous, lymph, cytosols, ascites, pleural effusions, amniotic fluid, bladder washes, and bronchioalveolar lavages, among other body fluid samples. The patient samples be fresh or frozen, and may be treated with heparin, citrate, or EDTA.

As an example of an immunoassay that may be used in the methods of the invention is a sandwich ELISA. However, it can be appreciated that other methods, in addition to those disclosed herein, may be used to quantify soluble VEGF-R2 protein in patient samples. Furthermore, a number of detection methods may be used to visualize the soluble VEGF-R2 protein, such as luminescent labels.

Many formats may be adapted for use with the methods of the present invention. For example, the detection and quantitation of soluble VEGF-R2 protein in patient samples may be performed, by enzyme-linked immunosorbent assays, radioimmunoassays, dual antibody sandwich assays, agglutination assays, fluorescent immunoassays, immunoelectron and scanning microscopy, among other assays commonly known in the art. The quantitation of soluble VEGF-R2 protein in such assays may be adapted by conventional methods known in the art. In one embodiment, serial changes in circulating soluble VEGF-R2 protein levels may be detected and quantified by a sandwich assay in which the capture antibody has been immobilized using conventional techniques on the surface of the support.

Suitable supports include, for example, synthetic polymer supports, such as polypropylene, polystyrene, substituted polystyrene, polyacrylamides (such as polyamides and polyvinylchloride), glass beads, agarose, and nitrocellulose.

An example of an ELISA sandwich immunoassay that may be used in the methods of the present invention, uses purified mouse anti-human soluble VEGF-R2 monoclonal antibody as the capture antibody and biotinylated goat anti-human soluble VEGF-R2 polyclonal antibody as the detector antibody. The capture monoclonal antibody is immobilized on microtiter plate wells. Diluted human serum/plasma samples or soluble VEGF-R2 standards (e.g., recombinant wild-type soluble VEGF-R2 protein) are incubated in the wells to allow binding of soluble VEGF-R2 antigen by the capture monoclonal antibody. After washing of wells, the immobilized soluble VEGF-R2 antigen is exposed to a biotinylated detector antibody after which the wells are again washed. A streptavidin-horseradish peroxidase conjugate is then added. After a final wash, TMB Blue Substrate is added to the wells to detect bound peroxidase activity. The reaction is stopped by the addition of 2.5 N sulfuric acid, and the absorbance is measured at 450 nm. Correlating the absorbance values of samples with the soluble VEGF-R2 standards allows the determination of a quantitative value of soluble VEGF-R2 in pg/ml of serum or plasma.

It can be appreciated that other proteins (e.g., inhibitors, oncoproteins, growth factor receptors, angiogenic factors, metastasis proteins, tumor markers, tumor suppressors, proteins associated with the VEGF pathway) may be suitable for detection and quantitation in combination with soluble VEGF-R2. For example, other proteins suitable for testing along with soluble VEGF-R2 include tissue inhibitor of metalloproteinase-1 (TIMP-1), HER-2/neu, ras p21, epidermal growth factor receptor (EGFR), platelet derived growth factor receptor alpha, vascular endothelial growth factor (VEGF), urokinase-type plasminogen activator (uPA), carcinoembryonic antigen (CEA), and p53. These other proteins may be detected using assays that are known to one of skill in the art. For example, immunoassays for the quantitation of HER-2/neu and TIMP-1 are commercially available, such as the Oncogene Science TIMP-1 ELISA (Oncogene Science, Cambridge, Mass. (USA)) which can detect ng/ml values of TIMP-1 levels in human serum or plasma.

Monitoring the pretreatment levels of soluble VEGF-R2 may be indicative of clinical outcome following treatment with a multi-kinase inhibitor (e.g., Sorafenib). One method of evaluating a clinical outcome may be assessment of response rate (RR), complete response (CR), partial response (PR), stable disease (SD), clinical benefit (including complete response (CR), partial response (PR), and stable disease (SD)), time to progression (TTP), progression free survival (PFS), and overall survival (OS).

The term “antibody” herein is used in the broadest sense and specifically covers monoclonal antibodies (including full length monoclonal antibodies), polyclonal antibodies, multispecific antibodies (e.g., bispecific antibodies), and antibody fragments. Antibodies useful according to the methods of the invention may be prepared by conventional methodology and/or by genetic engineering. For example, antibodies according to the invention include those antibodies that bind to soluble VEGF-R2.

“Antibody fragments” comprise a portion of a full length antibody, generally the antigen binding or variable domain thereof. Examples of antibody fragments include Fab, Fab′, F(ab′)₂, and Fv fragments; diabodies; linear antibodies; single-chain antibody molecules; biospecific antibodies; and multispecific antibodies formed from antibody fragments.

The term “monoclonal antibody” as used herein refers to an antibody obtained from a population of substantially homogeneous antibodies, that is, individual antibodies comprising an identical population except for possible naturally occurring mutations that may be present in minor amounts. Monoclonal antibodies are highly specific, that is, directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler, et al., (Nature 256:495, 1975), or may be made by recombinant DNA methods (see, e.g., U.S. Pat. No. 4,816,567). Monoclonal antibodies may also be isolated from phage antibody libraries using the techniques described in, for example, Clackson, et al., (Nature 352:624-628, 1991) and Marks, et al., (J. Mol. Biol. 222:581-597, 1991).

The monoclonal antibodies herein also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison, et al., Proc. Natl. Acad. Sci. USA 81:6851-6855, 1984).

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit, or nonhuman primate having the desired specificity, affinity, and capacity. In some instances, framework region (FR) residues of the human immunoglobulin may be replaced by corresponding non-human residues. Furthermore, humanized antibodies may comprise residues which are not found in the recipient antibody or in the donor antibody. Such modifications are made to further refine antibody performance. In general, the humanized antibody may comprise substantially all of at least one or typically two variable domains, in which all or substantially all of the hypervariable regions correspond to those of a non-human immunoglobulin and all or substantially all of the FRs are those of a human immunoglobulin sequence. The humanized antibody optionally also may comprise at least a portion of an immunoglobulin constant region (Fc), typically that of a human immunoglobulin. For a review, see Jones, et al., (Nature 321:522-525, 1986); Reichmann, et al., (Nature 332:323-329, 1988); and Presta, (Curr Op. Struct. Biol. 2:593-596, 1992).

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review, see Pluckthun (The Pharmacology of Monoclonal Antibodies, Vol. 113, Rosenburg and Moore eds. Springer-Verlag, New York, pp. 269-315, 1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger, et al., (Proc. Natl. Acad. Sci. USA 90:6444-6448, 1993).

The expression “linear antibodies” refers to the antibodies described in Zapata, et al., (Protein Eng. 8(10):1057-1062, 1995). Briefly, such antibodies comprise a pair of tandem Fd segments (V_{H}-C_{H}1-V_{H}-C_{H}1) which form a pair of antigen binding regions. Linear antibodies can be bispecific or monospecific.

Representative monoclonal antibodies useful according to this invention include mouse anti-human total soluble VEGF-R2 monoclonal antibodies designed to measure human soluble VEGF-R2 (e.g., Oncogene Sciences sandwich ELISA kit for VEGFR-165). Monoclonal antibodies useful according to this invention serve to identify soluble VEGF-R2 proteins in various laboratory prognostic tests, for example, in clinical samples.

General texts describing additional molecular biological techniques useful herein, including the preparation of antibodies include Berger and Kimmel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biology, (F. M. Ausabel et al. [Eds.], Current Protocols, a joint venture between Green Publishing Associates, Inc. and John Wiley & Sons, Inc. (supplemented through 2000)); Harlow et al., (Monoclonal Antibodies: A Laboratory Manual, Cold Spring Harbor Laboratory Press (1988), Paul [Ed.]); Fundamental Immunology, (Lippincott Williams & Wilkins (1998)); and Harlow, et al., (Using Antibodies: A Laboratory Manual, Cold Spring Harbor Laboratory Press (1998)).

The antibodies useful according to this invention to identify soluble VEGF-R2 proteins may be labeled in any conventional manner. An example of a label is horseradish peroxidase, and an example of a method of labeling antibodies is by using biotin-strepavidin complexes.

As appropriate, antibodies used in the immunoassays of this invention that are used as tracers may be labeled in any manner, directly or indirectly, that results in a signal that is visible or can be rendered visible. Detectable marker substances include radionuclides, such as ³H, ¹²⁵I, and ¹³¹I; fluorescers, such as, fluorescein isothiocyanate and other fluorochromes, phycobiliproteins, phycoerythin, rare earth chelates, Texas red, dansyl and rhodamine; colorimetric reagents (chromogens); electron-opaque materials, such as colloidal gold; bioluminescers; chemiluminescers; dyes; enzymes, such as, horseradish peroxidase, alkaline phosphatases, glucose oxidase, glucose-6-phosphate dehydrogenase, acetylcholinesterase, alpha -, beta-galactosidase, among others; coenzymes; enzyme substrates; enzyme cofactors; enzyme inhibitors; enzyme subunits; metal ions; free radicals; or any other immunologically active or inert substance which provides a means of detecting or measuring the presence or amount of immunocomplex formed. Exemplary of enzyme substrate combinations are horseradish peroxidase and tetramethyl benzidine (TMB), and alkaline phosphatases and paranitrophenyl phosphate (pNPP).

Another detection and quantitation systems according to this invention produce luminescent signals, bioluminescent (BL) or chemiluminescent (CL). In chemiluminescent (CL) or bioluminescent (BL) assays, the intensity or the total light emission is measured and related to the concentration of the unknown analyte. Light can be measured quantitatively using a luminometer (photomultiplier tube as the detector) or charge-coupled device, or qualitatively by means of photographic or X-ray film. The main advantages of using such assays is their simplicity and analytical sensitivity, enabling the detection and/or quantitation of very small amounts of analyte.

Exemplary luminescent labels are acridinium esters, acridinium sulfonyl carboxamides, luminol, umbelliferone, isoluminol derivatives, photoproteins, such as aequorin, and luciferases from fireflies, marine bacteria, Vargulla and Renilla. Luminol can be used optionally with an enhancer molecule such as 4-iodophenol or 4-hydroxy-cinnamic acid. Typically, a CL signal is generated by treatment with an oxidant under basic conditions.

Additional luminescent detection systems are those wherein the signal (detectable marker) is produced by an enzymatic reaction upon a substrate. CL and BL detection schemes have been developed for assaying alkaline phosphatases (AP), glucose oxidase, glucose 6-phosphate dehydrogenase, horseradish peroxidase (HRP), and xanthine-oxidase labels, among others. AP and HRP are two enzyme labels which can be quantitated by a range of CL and BL reactions. For example, AP can be used with a substrate, such as an adamantyl 1,2-dioxetane aryl phosphate substrate (e.g. AMPPD or CSPD; Kricka, L. J., “Chemiluminescence and Bioluminescence, Analysis by,” Molecular Biology and Biotechnology: A Comprehensive Desk Reference (ed. R. A. Meyers) (VCH Publishers; N.Y., N.Y.; 1995)); for example, a disodium salt of 4-methoxy-4-(3-phosphatephenyl) spiro [1,2-dioxetane-3,2′-adamantane], with or without an enhancer molecule such as 1-(trioctylphosphonium methyl)-4-(tributylphosphonium methyl) benzene diochloride. HRP is may be used with substrates, such as, 2′,3′,6′-trifluorophenyl-methoxy-10-methylacridan-9-carboxylate.

CL and BL reactions may be adapted for analysis not only of enzymes, but also of other substrates, cofactors, inhibitors, metal ions, and the like. For example, luminol, firefly luciferase, and marine bacterial luciferase reactions are indicator reactions for the production or consumption of peroxide, ATP, and NADPH, respectively. They may be coupled to other reactions involving oxidases, kinases, and dehydrogenases, and may be used to measure any component of the coupled reaction (enzyme, substrate, cofactor).

The detectable marker may be directly or indirectly linked to an antibody used in an assay of this invention. Exemplary of an indirect linkage of the detectable label is the use of a binding pair between an antibody and a marker or the use of a signal amplification system.

Examples of binding pairs that may be used to link antibodies to detectable markers are biotin/avidin, streptavidin, or anti-biotin; avidin/anti-avidin; thyroxine/thyroxine-binding globulin; antigen/antibody; antibody/anti-antibody; carbohydrate/lectins; hapten/anti-hapten antibody; dyes and hydrophobic molecules/hydrophobic protein binding sites; enzyme inhibitor, coenzyme or cofactor/enzyme; polynucleic acid/homologous polynucleic acid sequence; fluorescein/anti-fluorescein; dinitrophenol/anti-dinitrophenol; vitamin B12/intrinsic factor; cortisone, cortisol/cortisol binding protein; and ligands for specific receptor protein/membrane associated specific receptor proteins.

Various means for linking labels directly or indirectly to antibodies are known in the art. For example, labels may be bound either covalently or non-covalently. Exemplary antibody conjugation methods are described in Avarmeas, et al., Scan. J. Immunol. 8(Suppl. 7):7, 1978); Bayer, et al., Meth. Enzymol. 62:308, 1979; Chandler, et al., J. Immunol. Meth. 53:187, 1982; Ekeke and Abuknesha, J. Steroid Biochem. 11:1579, 1979; Engvall and Perlmann, J. Immunol. 109:129, 1972; Geoghegan, et al., Immunol. Comm. 7:1, 1978; and Wilson and Nakane, Immunofluorescence and Related Techniques, Elsevier/North Holland Biomedical Press; Amsterdam (1978).

Depending upon the nature of the label, various techniques may be employed for detecting and quantitating the label. For fluorescers, a large number of fluorometers are available. For chemiluminescers, luminometers or films are available. With enzymes, a fluorescent, chemiluminescent, or colored product may be determined or measured fluorometrically, luminometrically, spectrophotometrically, or visually.

Various types of chemiluminescent compounds having an acridinium, benzacridinium, or acridan type of heterocyclic ring systems are other examples of labels. Examples of acridinium esters include those compounds having heterocyclic rings or ring systems that contain the heteroatom in a positive oxidation state including such ring systems as acridinium, benz[a]acridinium, benz[b]acridinium, benz[c]acridinium, a benzimidazole cation, quinolinium, isoquinolinium, quinolizinium, a cyclic substituted quinolinium, phenanthridinium, and quinoxalinium.

The tracer may be prepared by attaching to the selected antibody either directly or indirectly a reactive functional group present on the acridinium or benzacridinium ester, as is well known to those skilled in the art (see, e.g., Weeks, et al., Clin. Chem. 29(8):1474-1479, 1983). Examples of compounds are acridinium and benzacridinium esters with an aryl ring leaving group and the reactive functional group present in either the para or the meta position of the aryl ring. (see, e.g., U.S. Pat. No. 4,745,181 and WO 94/21823).

As used herein, “VEGF pathway-directed therapies” include any therapies that are targeted to the VEGF pathway, including inhibition of VEGF protein expression (e.g., antisense oligonucleotides), prevention of membrane localization essential for VEGFR activation, or inhibition of downstream effectors of VEGFR (e.g., Raf serine/threonine kinases). VEGF pathway-directed therapies include multi-kinase inhibitors, tyrosine kinase inhibitors, monoclonal antibodies, and bis-aryl ureas.

An example of a kinase inhibitor is the bis-aryl urea Sorafenib, a small molecule and novel dual-action inhibitor of both Raf (a protein-serine/threonine kinase) and VEGFR (vascular endothelial growth factor receptor, a receptor tyrosine kinase), and consequently an inhibitor of both tumor cell proliferation and angiogenesis (Onyx Pharmaceuticals, Richmond, Calif., and Bayer Pharmaceuticals Corporation, West Haven, Conn. (USA); Lyons, et al., Endocrine-Related Cancer 8:219-225, 2001). In addition, Sorafenib has been found to inhibit several other receptor tyrosine kinases involved in tumor progression and neovascularization, including PDGFR-β, Flt-3, and c-KIT. PD166285 (Pfizer, Groton, Conn.), a general tyrosine kinase inhibitor, can antagonize both PDGF and FGF-2-mediated responses (Bansai, et al., J. Neuroscience Res. 74(4):486-493, 2003).

Other exemplary therapies that target the VEGF pathway include: Sutent/SU11248, PTK 787, MLN518, PKC-412, CDP860, and XL9999. Sutent/SU11248 (sunitinib malate; an indoline-2-one) (Pfizer, Groton, Conn.) targets receptor tyrosine kinases (RTKs) including PDGFR, with anti-angiogenic and anti-tumor effects. PDGFR plays a significant role in fostering angiogenesis by regulating the proliferation and migration of pericytes, cells that support blood vessels, and Sutent/SU11248 is believed to inhibit PDGFR's angiogenic action.

PTK 787 (Novartis, Basel, Switzerland and Schering AG, Berlin, Germany) is a oral small molecule anti-angiogenesis agent (anilinophthalazine) active against PDGFR, as well as against VEGFR and c-Kit tyrosine kinase receptors (see, e.g., Garcia-Echevera and Fabbro, Mini Reviews in Medicinal Chemistry 4(3):273-283, 2004).

MLN518 (formerly known as CT53518; Millenium Pharmaceuticals, Cambridge, Mass.) is an oral, small molecule designed to inhibit type III receptor tyrosine kinases (RTKs), including PDGFR, FLT3, and c-Kit.

PKC-412 [midostaurin; N-benzoyl-staurosporine (a derivative of staurosporine, a product of Streptomyces bacteria); Novartis, Basel, Switzerland) inhibits PDGFR, VEGFR and multiple protein kinase Cs, “which makes it especially attractive in patients with wild-type KIT with mutations in PDGFR” (PKC 412-An Interview with Charles Blanke, MD, FACP (www.gistsupport.org/pkc412.html); see also Reichardt, et al., J. Clin. Oncol. 23(16S):3016, 2005).

XL999 (one of several Spectrum Selective Kinase Inhibitors® (SSKIs) from Exelixis (South San Francisco, Calif., USA)] inhibits VEGFR, as well as other RTKs, such as PDGFR-beta, FGFR1, and FLT3.

### EXAMPLES

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Example 1

### Solid Phase Sandwich Microtiter ELISA for Human Serum and Plasma Sample Preparation

Suitable samples for analysis by the soluble VEGF-R2 ELISA include human plasma treated with heparin, citrate, or EDTA, and human serum. Due to possible interfering factors, special care must be taken in the preparation and assay of human serum and plasma. Any flocculant material should be removed from samples by microcentrifugation prior to dilution. The initial concentration of the serum or plasma specimen to be examined should be about 12-13% (a 1:8 dilution of specimen in sample diluent). For example, 40 μl of sample may be diluted into 280 μl of sample diluent, and 100 μl added to the microplate wells.

### Assay Procedure

The following ELISA protocol is that used for the sandwich ELISA (Oncogene Science, Cambridge, Mass.) to measure human VEGFR-165 in human plasma or serum.
1. Prepare a working solution (1X) of Platewash (Provided as part of the assay kit).
2. Add prediluted samples and Controls, and each of the six soluble VEGF-R2165 Standards (0 to 8000 pg/mL) in duplicate by pipetting 100 μL into the appropriate wells using clean pipet tips for each sample and Standard. Add Standard 0 to one additional well to be used for determination of Substrate blank.
3. Cover wells with clean plastic wrap or plate sealer. Incubate microtiter plate for 1.5 hours at 37° C.
4. Carefully remove the plastic wrap or plate sealer. Wash wells using 300 μL per well with six cycles of Platewash buffer (Wash for three cycles, rotate the plate 180°, and wash for three more cycles).
5. Pipet 100 μL of the Detector Antibody into all wells except the Substrate blank well, which is left empty. Cover the wells with a fresh piece of plastic wrap. Incubate microtiter plate for 1 hour at 37° C.
6. Prepare Working Conjugate by diluting an appropriate volume of Conjugate Concentrate (1:50 dilution) into Conjugate Diluent.
7. Wash wells as in Step 4. Proceed immediately to Step 8.
8. Pipet 100 μL of Working Conjugate into all wells except the Substrate blank well, which is left empty. Cover the wells with a fresh piece of plastic wrap. Incubate the microtiter plate at room temperature (20-27° C.) for 1 hour.
9. Prepare Working Substrate by combining equal parts of Solution A and Solution B.

Six mL of each Substrate solution will provide 12 mL of Working Substrate, sufficient to develop one microtiter plate. Adjust volume of Working Substrate based on number of strips used. Mix well.
10. Dispense Working Substrate into a clean reagent trough and allow it to come to room temperature.
11. Wash wells as in Step 5. CAUTION: Do not allow plates to dry out. Proceed immediately to Step 12.
12. Pipet 100 μL of Working Substrate into all wells and cover the plate with plastic wrap or plate sealer. Incubate the microtiter plate at room temperature (20-27° C.) for 45 minutes.
13. Pipet 100 μL of Stop Solution into all wells.
14. Measure absorbance in each well using a spectrophotometric plate reader at a wavelength of 650 nm. Wells should be read within 30 minutes of adding the Stop Solution.

### Standard Curves

Quantitative analyses were made by constructing a standard curve using soluble VEGF-R2 standard (e.g., recombinant human soluble VEGF-R2) at 6 different concentrations of 0, 150, 1000, 3000, 5000, and 8000 pg/ml.

### Human Serum and Plasma Samples

Frozen plasma samples were obtained from patients with confirmed renal cell carcinoma prior to treatment with Sorafenib.

### Example 2

### Plasma from Renal Cell Carcinoma Patients

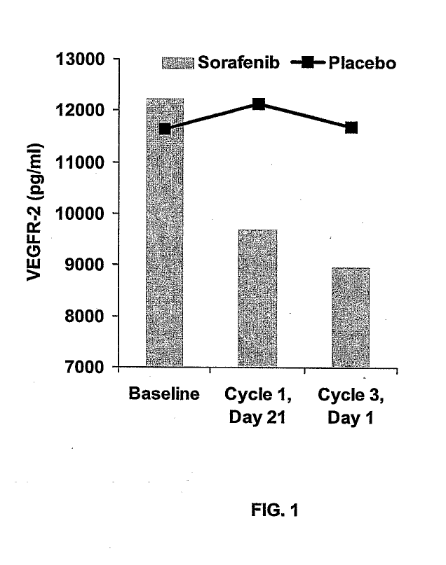

Duplicate samples were used to measure the soluble VEGF-R2 level using a soluble VEGF-R2 ELISA (R&D Systems, Minneapolis, Minn.) per the manufacturers directions. The mean value of the duplicate measurements was determined for each patient. The mean levels of soluble VEGF-R2 are reported in Table 1 for three time points, Baseline (pretreatment), Cycle 1 Day 21, and Cycle 3 Day 1 for both a group of patients treated with Sorafenib and a group of patients treated with a placebo. The same data is shown in FIG. 1. The results shown that the Sorafenib-treated patient group have soluble VEGF-R2 levels that decrease significantly from baseline (p<<0.01 using a paired t-test) at both time points, but this does not occur in the placebo-treated group (p>0.05).

TABLE 1
Soluble VEGFR-2

| | Median soluble VEGF-R2 (pg/ml) | | |
|---|---|---|---|
| | (Number of patients) | | |
| | | Cycle 1 | Cycle 3 |
| | Baseline | Day 21 | Day 1 |
| Sorafenib | 12237 | 9704 | 8964 |
| | (149) | (196) | (197) |
| Placebo | 11650 | 12128 | 11698 |
| | (102) | (128) | (132) |

The description of the foregoing embodiment of the invention has been presented for purposes of illustration and description. It is not intended to be exhaustive or to limit the invention to the precise form disclosed, and obviously many modifications and variations are possible in light of the above teachings. The embodiments were chosen and described in order to explain the principles of the invention and its practical application to enable thereby others skilled in the art to utilize the invention in various embodiments and with various modifications as are suited to the particular use contemplated. All references cited herein are hereby incorporated by reference.

1. A method for monitoring the status of a disease associated with the soluble VEGF-R2 pathway in a patient, and/or monitoring how a patient with said disease is responding to a therapy comprising immunologically detecting and quantifying serial changes in soluble VEGF-R2 protein levels in patient samples taken over time, wherein increasing levels of soluble VEGF-R2 protein over time indicate disease progression or a negative response to said therapy, and wherein decreasing levels of soluble VEGF-R2 protein over time indicate disease remission or a positive response to said therapy.

2. The method of claim 1, wherein said therapy is selected from multi-kinase inhibitors, tyrosine kinase inhibitors, monoclonal antibodies, and bis-aryl ureas.

3. The method of claim 1, wherein said therapy is a VEGF pathway-directed therapy.

4. The method of claim 3, wherein said VEGF pathway-directed therapy is the tyrosine kinase inhibitor imatinib mesylate or the bis-aryl urea Sorafenib.

5. The method of claim 1, wherein said disease is a preneoplastic/neoplastic disease.

6. The method of claim 5, wherein said preneoplastic/neoplastic disease is selected from the group consisting of metastatic medulloblastoma, dermatofibrosarcoma protruberans, gastrointestinal stromal tumors, colorectal cancer, colon cancer, lung cancer, non-small-cell lung cancer, small-cell lung cancer, chronic myeloproliferative diseases, acute myelogenous leukemia, thyroid cancer, pancreatic cancer, bladder cancer, kidney cancer, melanoma, breast cancer, prostate cancer, ovarian cancer, cervical cancer, head-and-neck cancer, brain tumors, hepatocellular carcinoma, hematologic malignancies, and precancers leading to the aforementioned cancers.

7. The method of claim 1 which is further prognostic for said disease, wherein said levels of soluble VEGF-R2 protein in the patient's samples are indicative of a better or poorer prognosis for said patient.

8. The method of claim 7, wherein said prognosis is a clinical outcome selected from the group consisting of response rate (RR), complete response (CR), partial response (PR), stable disease (SD), time to progression (TTP), progression free survival (PFS), overall survival (OS), and clinical benefit, which comprises complete response (CR), partial response (PR), and stable disease (SD).

9. The method of claim 1, wherein said patient's samples are pretreatment samples.

10. The method of claim 1, wherein said patient sample is selected from the group consisting of blood, serum, plasma, urine, saliva, semen, breast exudate, cerebrospinal fluid, tears, sputum, mucous, lymph, cytosols, ascites, pleural effusions, amniotic fluid, bladder washes and bronchioalveolar lavages.

11. The method of claim 1, wherein said patient sample is serum or plasma.

12. The method of claim 1, wherein said immunological detection and quantitation is by an immunoassay in the form of a sandwich ELISA or equivalent assay.

13. The method of claim 12, wherein the sandwich ELISA or equivalent assay comprises the use of one or more monoclonal antibodies that selectively bind the soluble VEGF-R2 protein.

14. The method of claim 1, further comprising the use of an immunoassay to detect or detect and quantify levels of one or more other proteins in the patient's samples.

15. The method of claim 14, wherein said other protein is or said other proteins are selected from the group consisting of inhibitors, oncoproteins, growth factor receptors, angiogenic factors, metastasis proteins, tumor markers, and tumor suppressors.

16. The method of claim 15 wherein said inhibitor is tissue inhibitor of metalloproteinase-1 (TIMP-1), said oncoproteins are selected from the group consisting of HER-2/neu and ras p21, said growth factor receptors are selected from the group consisting of epidermal growth factor receptor (EGFR) and platelet derived growth factor receptor alpha (PDGFR-α), said angiogenic factor is vascular endothelial growth factor (VEGF), said metastasis protein is urokinase-type plasminogen activator (uPA), said tumor marker is carcinoembryonic antigen (CEA), and said tumor suppressor is p53.

17. A method of therapy selection for a human patient with a disease, comprising:
(a) immunologically detecting and quantifying the average level of soluble VEGF-R2 protein in control samples taken from individuals of a control population;
(b) immunologically detecting and quantifying serial changes in soluble VEGF-R2 protein levels in equivalent patient samples taken from the patient over time;
(c) comparing the levels of soluble VEGF-R2 protein in the patient's samples to the average level of soluble VEGF-R2 protein in the control samples; and
(d) determining whether to use conventional therapy and/or VEGF pathway-directed therapy to treat the patient based upon the differences between the levels of soluble VEGF-R2 protein in the patient's samples and the average level of soluble VEGF-R2 protein in the control samples, and in view of the serial changes among the levels of soluble VEGF-R2 protein in the patient's samples.

18. The method of claim 17, wherein said patient's samples are pretreatment samples.

19. The method of claim 17 which is further prognostic for said disease, wherein said levels of soluble VEGF-R2 protein in the patient's samples are indicative of a better or poorer prognosis for said patient.

20. The method of claim 19, wherein said prognosis is a clinical outcome selected from the group consisting of response rate (RR), complete response (CR), partial response (PR), stable disease (SD), time to progression (TTP), progression free survival (PFS), overall survival (OS), and clinical benefit, which comprises complete response (CR), partial response (PR), and stable disease (SD).

21. The method of claim 17, wherein said disease is a preneoplastic/neoplastic disease.

22. The method of claim 21 which said preneoplastic/neoplastic disease is selected from the group consisting of metastatic medulloblastoma, dermatofibrosarcoma protruberans, gastrointestinal stromal tumors, colorectal cancer, colon cancer, lung cancer, non-small-cell lung cancer, small-cell lung cancer, chronic myeloproliferative diseases, acute myelogenous leukemia, thyroid cancer, pancreatic cancer, bladder cancer, kidney cancer, melanoma, breast cancer, prostate cancer, ovarian cancer, cervical cancer, head-and-neck cancer, brain tumors, hepatocellular carcinoma, hematologic malignancies, and precancers leading to the aforementioned cancers.

23. The method of claim 17, wherein the patient samples are from a cancer patient who has not responded to treatment.

24. The method of claim 17, further comprising the use of an immunoassay to detect or detect and quantify levels of one or more other proteins in the subject's samples.

25. The method of claim 24, wherein said other protein is or said other proteins are selected from the group consisting of inhibitors, oncoproteins, growth factor receptors, angiogenic factors, metastasis proteins, tumor markers, and tumor suppressors.

26. The method of claim 25 wherein said inhibitor is tissue inhibitor of metalloproteinase-1 (TIMP-1), said oncoproteins are selected from the group consisting of HER-2/neu and ras p21, said growth factor receptors are selected from the group consisting of epidermal growth factor receptor (EGFR) and platelet derived growth factor receptor alpha (PDGFR-α), said angiogenic factor is vascular endothelial growth factor (VEGF), said metastasis protein is urokinase-type plasminogen activator (uPA), said tumor marker is carcinoembryonic antigen (CEA), and said tumor suppressor is p53.

27. A diagnostic method to detect a disease associated with a VEGF pathway in a patient comprising:
(a) immunologically detecting and quantifying the average level of soluble VEGF-R2 protein in control samples taken from individuals of a control population;
(b) immunologically detecting and quantifying serial changes in soluble VEGF-R2 protein in samples of a patient sample taken from a patient over time; and
(c) comparing the levels of soluble VEGF-R2 protein in the patient's samples to the average level of soluble VEGF-R2 protein in the control samples;
wherein a level of soluble VEGF-R2 protein in the patient's samples that is above the average level of soluble VEGF-R2 protein in the control samples is indicative of an activated VEGF pathway and the presence of disease in the patient.

28. The method of claim 27, wherein said immunological detection and quantification of steps (a) and (b) is by an immunoassay in the form of a sandwich ELISA or equivalent assay.
